(19) United States
(12) Patent Application Publication
(10) Pub. No.: US 2002/0138131 A1
(43) Pub. Date: Sep. 26, 2002

(54) Rail stent

(76) Inventors: Kenneth S. Solovay, Weston, FL (US); Thomas P. Jacobs, Fort Lauderdale, FL (US)

Correspondence Address: BANNER & WITCOFF, 1001 G STREET N W, SUITE 1100, WASHINGTON, DC, 20001, US

(21) Appl. No.: 10/100,986

(22) Filed: Mar. 20, 2002

Related U.S. Application Data
(60) Provisional application No. 60/276,913, filed on Mar. 20, 2001.

(51) Int. Cl. A61F 2/06

(52) U.S. Cl. 623/1.15

(57) ABSTRACT

A stent with a plurality of support elements that are deployable within a body for supporting a vessel or other body structure. The stent includes first and second terminal ends and a length extending between the terminal ends. Support rails extend between the terminal ends and through the support members in a direction parallel to the longitudinal axis of the stent. The support elements can include openings for receiving the rails. The rails can be formed of a spring so that the stent can easily conform to the minor bend of a curved vessel when the stent is deployed.

The present application claims the benefit and priority of Provisional U.S. Application Serial No. 60/276,913 filed on Mar. 20, 2001, the full disclosure of which is incorporated herein by reference.

### FIELD OF THE INVENTION

The present invention relates to a stent for use in supporting vascular tissue, and particularly, to a stent having improved longitudinal structural flexibility.

### BACKGROUND OF THE INVENTION

It is generally known to insert a resiliently expansible stent into a blood vessel to provide radial hoop support within the vessel in the treatment of atherosclerotic stenosis. For example, it is generally known to open a blocked cardiac blood vessel by known methods (e.g., balloon angioplasty or laser ablation) and to keep that blood vessel open using such a stent. These stents are generally formed of a biocompatible material, such as stainless steel, and have slots or holes cut therein so a balloon can expand the stent after being deployed into the blood vessel

However, a conventional stent structure tends to be longitudinally inflexible (i.e., along a length of the stent), and therefore tends to be resistant to transverse deformation. As a result, the conventional stent tends to straighten a blood vessel into which it is inserted because it resists conforming to the shape of a curved blood vessel path. Currently, there is some discussion in the art regarding a relationship between this tendency to straighten a blood vessel and the onset of restensosis (i.e., blood vessel reclosure).

Conventional, longitudinally inflexible stents are disclosed in, for example, U.S. Pat. No. 6,113,628 to Borghi and U.S. Pat. No. 5,653,727 to Wiktor. The stents discussed in these patents are not capable of achieving the longitudinal flexibility needed to prevent restenosis. These stents include circumferential support hoops that are securely spaced from each other and from the ends of the stent so that they do not experience relative axial movement. The spacing between adjacent hoops is maintained by rigid connections or bridge elements (sometimes referred to in the art as “bridges”) between adjacent support hoops and a rigid connection between each support hoop and at least one longitudinal rail that extends from a first end of the stent to a second end of the stent. This type of secure, rigid spacing prevents the support hoops from moving longitudinally along the rail(s) of the stent and prevents the stent from conforming to the curvature of the blood vessel in which it is deployed.

It is known to use a stent for controlled, time release therapeutic agent delivery within a vessel. For example, this concept is discussed in U.S. Pat. No. 5,102,417 to Palmaz and U.S. Pat. No. 5,464,650 to Berg, et al., both of which are hereby incorporated by reference in their entirety. These patents disclose different methods for applying agents, including therapeutic drugs, to a stent in order to reduce the incidence of restenosis, increase vascular healing and/or treat various conditions within the body in which the stent is deployed. However, the coatings of these stents are typically interrupted when the stent is expanded, thereby limiting their effectiveness. Additionally, these stents and the coatings used to carry these agents can be very expensive to manufacture.

### SUMMARY OF THE INVENTION

The present invention is directed to an intraluminal stent having increased longitudinal flexibility when compared to prior art stents. Longitudinal flexibility as used herein relates to the flexibility of the stent structure (or portions thereof) to move relative to its major, longitudinal axis of extension.

An example of a stent according to the present invention includes a helically wound stent element freely mounted on a plurality of flexible rail elements that extend along the length of the stent. Because the stent element is freely mounted on the rail elements, various portions of the stent element are free to slide along the rail elements. Therefore, when the stent is placed in situ in a curved or otherwise bent blood vessel, the increased longitudinal flexibility of portions of the stent element along the axial extent of the stent allows those stent portions on the inside of the curve to move freely towards each other. On the other hand, the corresponding stent portions on the outside of the curve are free to move apart from each other. In this manner, the stent can more easily conform to the bend in the blood vessel and reduce the tendency of the stent to straighten the blood vessel.

In another embodiment of the present invention, the stent includes a plurality of independent hoop elements freely mounted on rail elements. The behavior of the hoop elements is similar to that of the helically wound stent structure as discussed above when the stent is bent or curved.

In yet another embodiment, the stent includes a plurality of hoop elements each connected to at least one adjacent hoop element and freely mounted on rail elements. This provides a consistent structural arrangement of the hoop elements along the extent of the stent while still providing increased longitudinal flexibility in accordance with the present invention.

In still another embodiment, a stent according to the present invention comprises first and second terminal ends and a length extending between these terminal ends. The stent also includes at least one support rail extending between the terminal ends and a plurality of circumferentially extending support elements. The support elements each have an opening that receives the at least one support rail so that the support elements are moveable relative to the rail between the terminal ends of the stent.

The number of openings within each support element can correspond with the number of rails extended through the stent. The number and position of the rails is chosen so that the friction characteristic between the support elements and the interior of the blood vessel will be negligible when the relative direction of motion is in line with the bends, but increases when against the edge of the bent sections.

The present invention can include a lengthwise hole design with a solid wire rail, a flexible coil rail with a bent node stent or a combination of the two. The present invention also incorporates the use of a flexible coil as the rail, allowing rail expansion or contraction and prevents the ends of the rail from protruding past the ends of the stent, especially along the minor (inside) radius of curvature of a vessel. The present stent has a smooth profile. It also allows for reduced wall thickness and a smaller profile when compared to conventional stents, thereby allowing for less traumatic navigation through arteries, such as those of a human. The closed loop rail stents of the present invention retain the rails and provide even spacious distribution of the inter-ring linkages.

### BRIEF DESCRIPTION OF THE DRAWINGS

The present invention will be even better understood with reference to the attached drawings, in which:

FIG. 1 illustrates a plurality of hoop elements according to the present invention;

FIG. 2 illustrates structural parameters of a respective hoop element according to the present invention that can be adjusted to provide different operational behaviors;

FIG. 3 illustrates the hoop elements of FIG. 1 mounted on rail elements according to the present invention;

FIGS. 4a-4c illustrate different geometries of the hoop elements according to the present invention;

FIGS. 5a and 5b illustrate hybrid combinations of geometries of the hoop elements according to the present invention;

FIG. 6 illustrates a variant geometry for the hoop elements according to the present invention, compared to that illustrated in FIG. 4a;

FIG. 7 illustrates an embodiment of the present invention including hoop elements, adjacent ones of which are joined together by at least one bridge element;

FIG. 8 illustrates a helically wound stent element mounted on rail elements according to the present invention;

FIG. 9 is an elevational view of a stent including a helically wound stent element;

FIG. 10 is a perspective view of a stent according to the present invention including a plurality of hoop elements;

FIGS. 11a-11c illustrate various examples of rail end structures for preventing hoop elements from becoming disengaged or dismounted from rail elements according to the present invention;

FIG. 12 is a perspective view of the stent according to another embodiment of the present invention;

FIG. 13 is a side view of the stent of FIG. 12;

FIG. 14 is a perspective view of the stent illustrated in FIG. 12;

FIG. 15 is an end view of the stent of FIG. 12;

FIG. 16 is a side view of the stent shown in FIG. 12; and

FIG. 17 is a cross section of a rail according to the present invention.

### DETAILED DESCRIPTION OF THE INVENTION

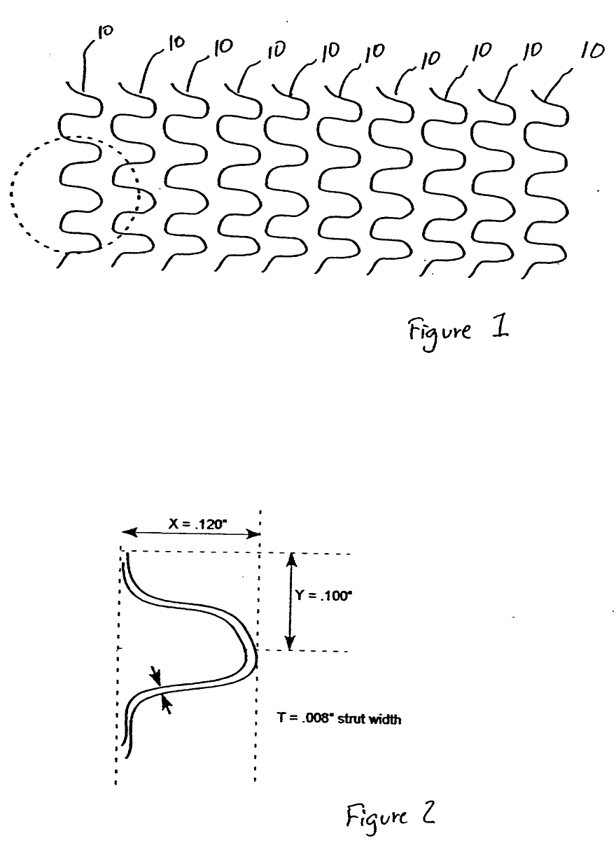

Referring to the figures where like numerals indicate the same element throughout the views, FIG. 1 illustrates a representative structure of hoop elements 10 for forming a portion of a stent 1 (see, for example, FIG. 3) according to the present invention. Each hoop element 10 is generally annular in shape. However, for the purpose of illustration, each hoop element 10 is depicted two-dimensionally on the paper as though it has been cut and laid flat.

Each hoop element 10 is made from a flexible, biocompatible material (i.e., from a material that is, for example, non-reactive and/or non-irritating). In one example of the present invention, hoop elements 10 are made from medical-grade metal wire formed as a closed loop (i.e., as an annular hoop) in a known manner, including, for example, micro-welding two ends of a wire segment together. Stainless steel, metal alloys, and polymeric materials used in conventional stents are representative examples of materials from which hoop elements 10 can be formed. The polymers for hoop elements 10 may, for example, be bioabsorbable polymers so that the stent can be absorbed into the body instead of being removed. As discussed below, these materials also include super elastic alloys such as Nitinol.

Preferably, each hoop element 10 has a sinusoidal or otherwise undulating form, such as the rounded wave shape seen in FIG. 1 by way of example. As shown in FIG. 1, the undulating form of the hoop elements includes peaks 12 and troughs 13 (space behind the peaks). Each peak points in a direction that is opposite that of the immediately, circumferentially positioned peak 11. The same is true of the valleys 13. The direction of undulation may be axial, as illustrated in FIG. 1, or radial, as seen, for example, in FIG. 10.

As seen in FIG. 2, certain parameters of hoop elements 10 can be altered in order to adjust the operational behavior of the stent 1. For example, as seen by way of example in FIG. 2, the wave height X and the peak-to-trough distance Y can be made larger or smaller, and/or the thickness T of the hoop element 10 can be made thicker or thinner. For example, X may be about 0.120 inch, Y may be about 0.100 inch, and T may be about 0.008 inch. However, other dimensions can also be used depending on the needs of the particular stent.

FIG. 3 illustrates hoop elements 10 freely mounted on rail elements 12. Rail elements 12 are desirably sufficiently flexible to accommodate bends, curves, etc. in a blood vessel. Rail elements 12 may be made from, for example and without limitation, metals, metallic alloys, glass or acrylic, and polymers. In contrast to bridge elements 28 which are generally the same thickness and the hoops 10 that they join and thus relatively inflexible, the thickness of the rail elements 12 can be designed to provide a desired degree of flexibility to a given stent.

As seen in FIG. 3, each rail element 12 is “woven” between adjacent hoop elements 10. In particular, each rail element 12 passes alternately inside and outside (or over and under, as seen in this depiction) adjacent hoop elements 10. Each rail element may, for example, be passed inside/outside (or under/over) adjacent hoop elements 10 at the respective peaks thereof, as illustrated in FIG. 3. Thus, adjacent longitudinally extending rail elements alternate inside and outside of a given hoop element 10 along a circumferential go direction thereof. This increases the structural integrity of the stent and helps resist lateral crushing forces that may be applied to the stent.

At least some of rail elements 12 may include end structures 14 for preventing the hoop elements 10 from unintentionally passing beyond the ends of the stent 1. End structures 14 may have several forms as illustrated in FIGS. 11a-11c. For example, end structures 14 may be mechanical stop members mounted on the ends of each rail element 12 to block the freely mounted hoop elements 10 from being dismounted from rail elements 12, effectively keeping the hoop elements 10 from “falling off” of the ends of rail elements 12. Examples of mechanical stop elements include balls or other protrusions formed at the ends of each rail element 12 that act as stops (see, for example, FIG. 11a).

Alternative mechanical stop elements include slotted members at the end of each rail as shown in FIG. 11c. In each of the above-discussed embodiments, all of the hoop elements 10 are free to move along the length of the rail elements 12, to the extent permitted by the mechanical stop elements.

In another example, end structures 14 may be a mechanical grasp structure by which the endmost hoop elements 10 are fixed in place relative to the ends of rail elements 12 (although the remaining hoop elements 10 remain freely mounted on rail elements 12). See, for example, FIG. 11b.

End structures 14 may also be (depending on their intended effect), a suture or other ligature by which a portion of an endmost hoop element 10 is tied to the end of rail element 12, or a weld (made by, for example, a laser) for bonding a portion of an endmost hoop element 10 to an end of rail element 12.

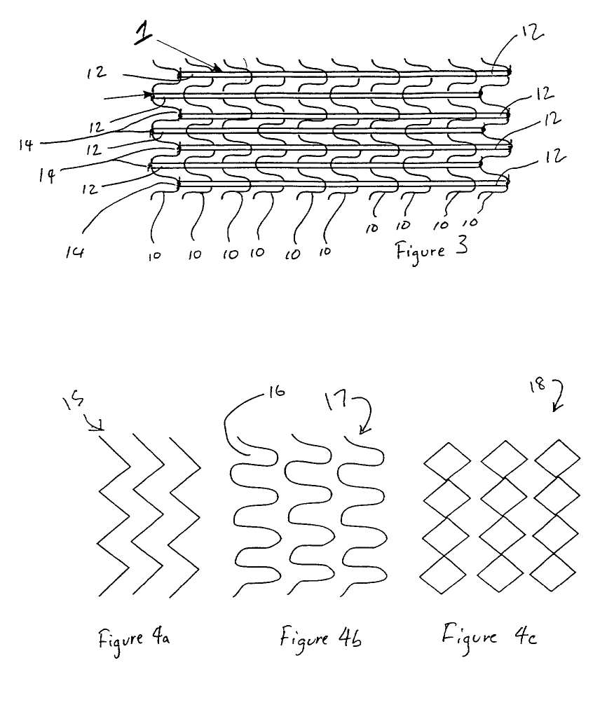

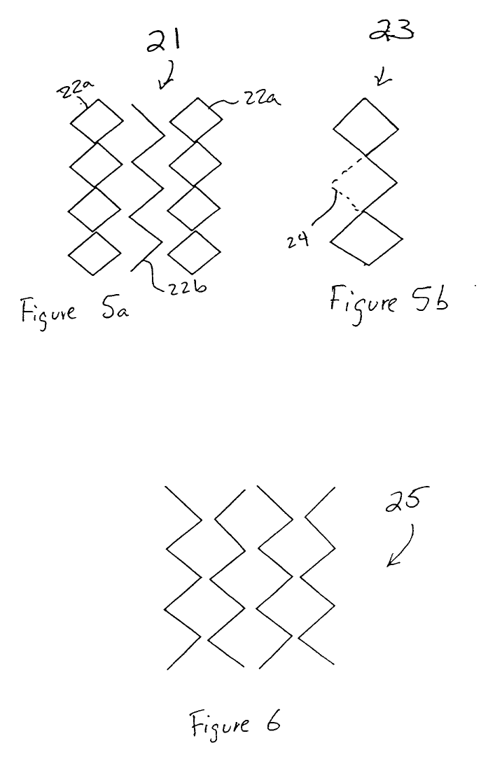

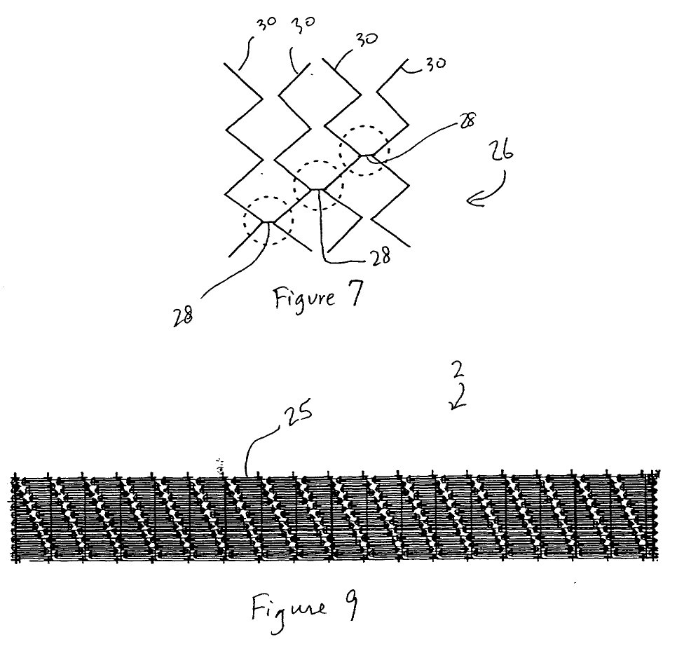

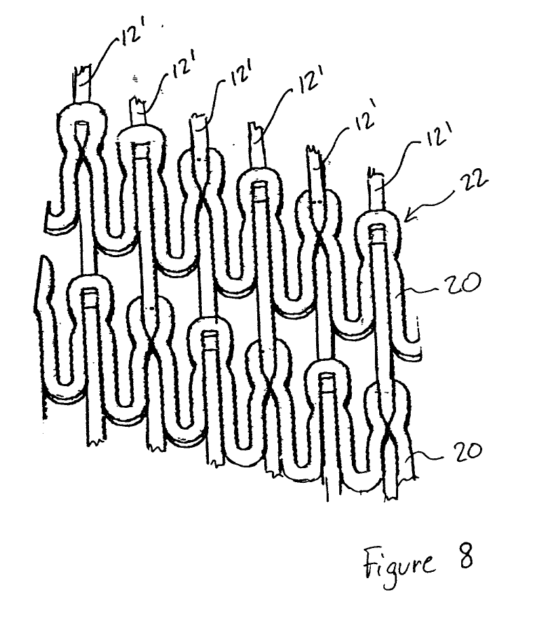

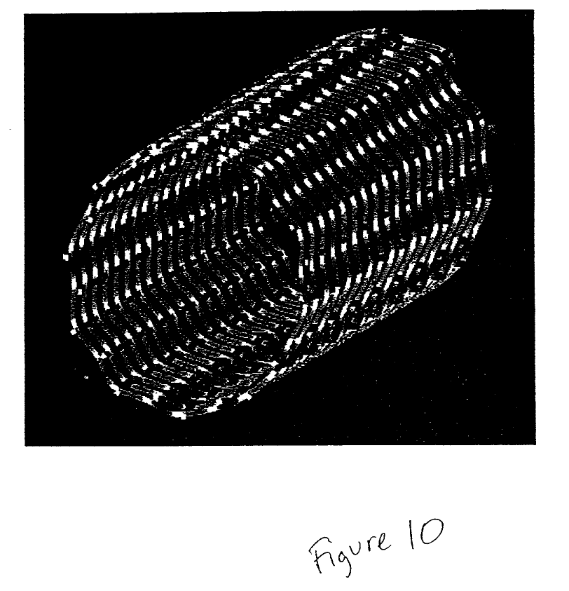

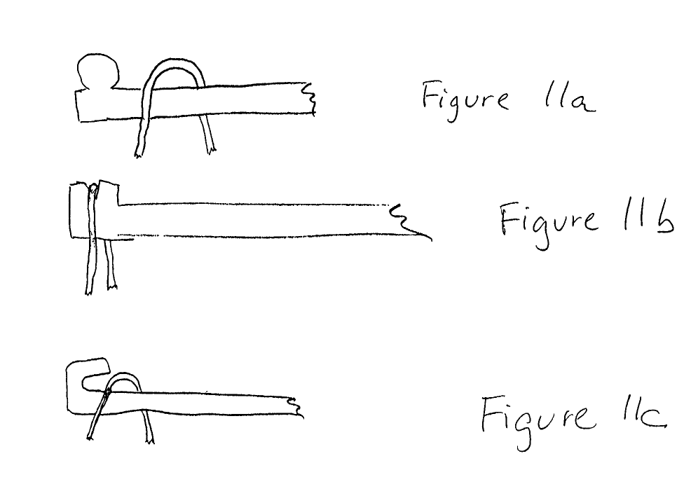

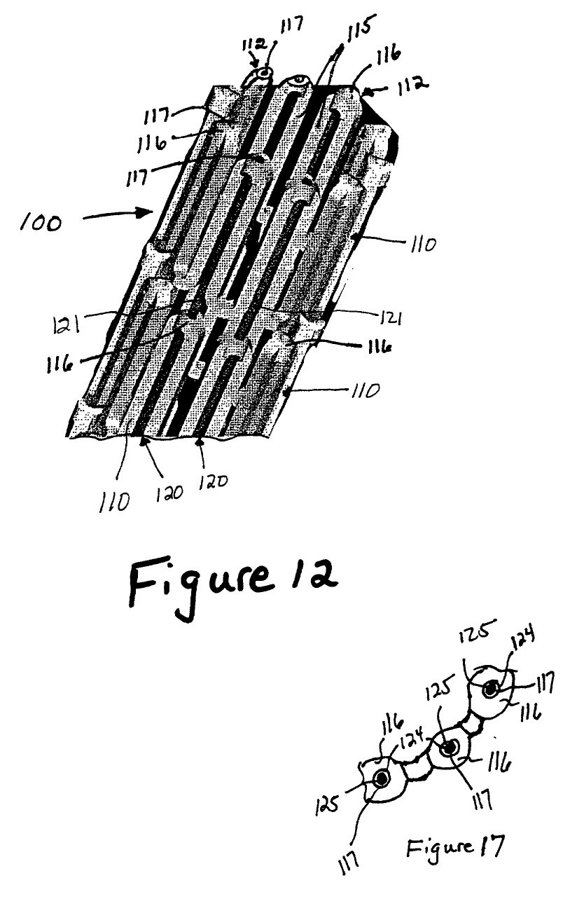

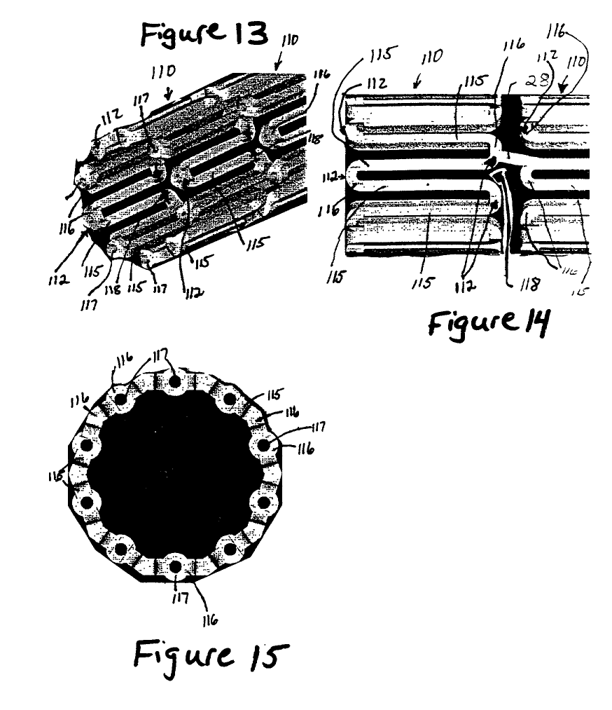

FIGS. 4a-4c, 5a-5b, 6, and 8 illustrate examples of geometries for the hoop elements 10. The geometries illustrated have different advantages.

For example, the geometry 15 illustrated in FIG. 4a is useful for forming hoop elements in self-expanding Nitinol stents, because it allows better crimping in preparation for insertion.

The geometry in FIG. 4b, which its relative wide “trough” portions 16 may, for example, facilitate engagement with a respective rail element 12 in the manner discussed above.

The diamond-shaped geometry 18 in FIG. 4c could be considered a variant of the sawtooth geometry shown in FIG. 4a. Like the example shown in FIG. 4a, the diamond-pattern in FIG. 4c is useful for self-expanding Nitinol stents because it facilitates crimping. In addition, it offers increased torsional rigidity and greater surface structure for support.

The geometries 21 and 23 of FIGS. 5a-5b, respectively, may, for example, be useful in covered stents or in stent-grafts requiring less scaffolding. Here, “scaffolding” refers to the amount of supporting structure in a given portion of the stent. For example, the combination of two diamond-shaped hoop elements 22a plus a sawtooth hoop element 22b does not provide as much supporting structure as three diamond-shaped hoop elements. Likewise, a diamond-shaped geometry 22 having part of some of the diamonds omitted (as indicated in phantom at 24) provides less supporting structure than hoop elements including complete diamonds.

The geometry 25 illustrated in FIG. 6 appears to have comparatively increased longitudinal flexibility and may permit specialized interaction with rail elements 12 in terms of force distribution and the like.

The geometry 26 illustrated in FIG. 7 includes at least one bridge element 28 between adjacent hoop elements 30, instead of providing a plurality of hoop elements that are independent from one another, as discussed above. Providing at least one bridge element 28 between adjacent hoop elements increases the structural integrity of the stent because it helps to keep the hoop elements 30 distributed along the length of the stent while still offering increased longitudinal flexibility.

Preferably, only a limited number of bridge elements 28 are provided between respective adjacent hoop elements. If too many bridge elements 28 are provided between adjacent hoop elements, the coupling therebetween becomes similar to providing a rigid coupling therebetween, such that the desired longitudinal flexibility according to the present invention is lost. By providing only a limited number of bridge elements 28 (including, without limitation, one bridge element 28), the resultant assembly can still provide a good approximation of using completely independent hoop elements.

Furthermore, the peripheral location at which bridge element(s) 28 are provided between respective adjacent hoop elements has an effect on longitudinal flexibility. For example, if two bridge elements are provided between a respective pair of adjacent hoop elements at diametrically opposite sides of the hoop elements, then, generally, the longitudinal flexibility therebetween is at a maximum at diametrically opposite sides of the hoop elements located at about 90 degrees from the bridge elements, and decreases along the circumference of the hoop elements in a direction approaching the respective bridge elements.

For the foregoing reasons, it may be useful or otherwise beneficial to provide, for example, one bridge element 28 between adjacent hoop elements 30, as illustrated in FIG. 8. Furthermore, it may be additionally useful to offset each bridge element 28 from an adjacent bridge element 28 along a circumferential direction, as is also illustrated in FIG. 7. This circumferential offset provides the structural integrity benefits of using a bridge element 28, but distributes the resultant restriction in longitudinal flexibility so that no one transverse direction of stent deflection is overly restricted.

As mentioned above, instead of using independent hoop elements 10, a single helically wound stent element 20 can be freely mounted on one or more substantially parallel rail elements 12′ as seen in FIG. 8. As with the hoop elements 10, rail elements 12′ are woven over/under respective portions of the stent element 20, such as, for example, over/under the respective peak portions.

FIG. 8 also illustrates a feature of the invention that is applicable to both the hoop elements 10 and the helically wound stent element 20. Specifically, a portion of, for example, stent element 20 adjacent to a respective peak portion is pinched in, or necked in, and a respective rail element 12′ is passed through the restricted portion 22 defined thereby. This advantageously limits relative movement between stent element 20 and rail element 12′. This maintains the relative alignment of rail elements 12′ and, as a result, increases the structural integrity and the overall hoop strength of the stent. It will be appreciated that instead of a pinched or a necked portion 22, an end portion of each peak portion could simply have a suitably sized hole formed therethrough (not shown here).

As mentioned above, the concept of a restricted portion 22, as seen in FIG. 8, is equally applicable to the arrangement of, for example, FIG. 3.

FIG. 9 is view of an entire stent 2 using a single helically wound stent element 25. It can be appreciated from FIG. 9 that a helically wound stent element, such as that illustrated at 25, has some effective similarity to a plurality of obliquely extending independent hoop elements. However, instead of using bridge elements (in the manner discussed with respect to FIG. 7), the use of a single stent element addresses at least some of the issues raised above with respect to longitudinal structural integrity.

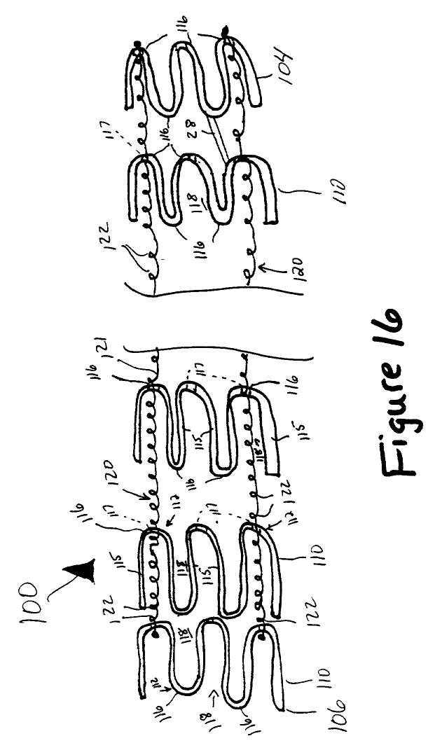

An additional embodiment of the stent 100 according to the present invention is illustrated in FIGS. 12-16. Like the embodiments discussed above and illustrated in FIGS. 3 and 8, the stent 100 illustrated in FIGS. 12-16 includes a plurality of support elements 110 spaced along its length. These support elements 110, like those discussed above 10, provide support to a blood vessel after the stent 100 has been deployed into a mammalian body and expanded. As with the other stents discussed above, stent 100 can be expanded by conventional techniques such as an inflatable balloon positioned within the stent 100.

As seen in FIGS. 12-15, the support elements 110 have the same general shape as those discussed above. Support elements 110 are generally annular in shape and have a generally hoop-like appearance. Hence, support elements 110 will be referred to as hoop elements 110 below. Adjacent hoop elements 110 are spaced from each other by bridge element 28 in the same manner as discussed above. Also, each hoop element 110 is formed of a flexible, biocompatible material such as those discussed above. As with the other stents, the stent 100 can be formed of a metal, metal alloy such as Nitinol, or polymer, etc.

As seen in FIGS. 12-14, the hoop elements 110 have a generally sinusoidal or otherwise undulating form. As shown in FIGS. 13 and 14, the undulating form of the hoop elements 110 is comprised of a plurality of substantially longitudinal struts 115 and a plurality of curved connecting members 116. Each curved member 116 connects adjacent longitudinal struts 115 together to form the continuous hoop element 110. Each curved connecting member 116 forms a peak 112 along the alternating path of each hoop 110. A trough 118 is formed at the end of each longitudinal strut 115 opposite the peak 112. Troughs 118 include the open spaces between adjacent longitudinal struts 115 that are connected to the same curved member 116 at a respective peak 112. As seen in FIG. 12, each peak 112 points in a direction that is opposite to that of the immediately proceeding peak 112 along the circumference of each hoop. Conversely, each peak 112 points in the same direction as the adjacent longitudinally spaced peak 112. The same is true of the troughs 118. For example, the troughs 118 are open in a direction opposite that of the immediately adjacent troughs 118 around the circumference of the hoop 110.

As with the other embodiments discussed above, stent 100 also includes at least one rail element 120 (hereinafter “rail”) that extends from a first terminal end 104 to a second terminal end 106. Each end 104, 106 is formed by one of the hoop elements 110 secured to the rail(s) 120. As illustrated in FIG. 12, the stent 100 can include two rails 120 that extend between the ends 104, 106. It is also contemplated that any number of rails 120 up to the number of peaks 112 along a hoop element 110 could be used. For example, if the hoop elements 110 include ten peaks 112, then up to ten rails 120 could be used. In between the hoops 110 at the terminal ends 104, 106, the remaining hoops 110 that are connected to each other by the bridge elements 28 are free to move along the rail(s) 120. These remaining hoops 110 slide along the rail(s) 120 so that the stent 100 can conform to the shape of the blood vessel.

Unlike the hoop elements 10, the hoop elements 110 include apertures 117 in the curved members 116 through which the rails 120 extend as shown in FIG. 12. Apertures 117 extend through the peaks 112 in a direction that is substantially parallel to the length of the stent 100. These apertures 117 retain and orient the supporting rail(s) 120 in a direction parallel to the length of the stent 100. Also, the rails 120 are completely contained within the walls (within the outer surface) of the stent 100. These walls form the apertures 117. By positioning the rails 120 extending within the walls of the stent 100, the rails 120 are not alternately woven from an inside surface to an outside surface of the stent 100 or in another way that could compromise the straightness of the rail 120.

In an embodiment of stent 100, the rail 120 is made of a flexible coil spring 121 instead of a solid wire. The flexible coil spring 121 is coiled about an axis that extends parallel to the longitudinal axis of the stent 100 before it is deployed within a blood vessel. When the stent 100 is straight, the coil spring 121 is at rest. As a result, the coil spring 121 is not under tension and no longitudinal pressure is applied to the hoops 110 by the coil spring 121. However, the coils 122 of the coil spring rails 121 are spaced from each other along the length of the stent 100 so that the coils 122 of the spring 121 can collapse upon themselves and shorten when and where needed. For example, when the stent 100 is deployed into a curved vessel, the stent 100 will conform to the curve of the blood vessel without straightening the vessel. This is accomplished by the coils 122 along the minor curve of the vessel compressing to a shorter length than when the stent 100 is at its rest length. The coil spring 121 aids in providing the shortest possible stent length on the minor radius of curvature of the vessel. Along the major curve, the coil spring 121 remains at rest or expands, and allows the stent 100 to follow the curve of the vessel.

In an alternative embodiment, the coil spring 121 is slightly extended and under tension when straight before deployment. As a result, the stent 100 is under a slight compressive force prior to deployment. This slightly compressive force assists in the stent 100 conforming to the minor curve of the vessel. In either of the above embodiments, the hoops 110 are spaced and held relative to each other by the bridge elements 28. In the second embodiment, the bridge elements 28 prevent the collapse of the stent 100 under the pressure of the coil spring 121.

In another embodiment, a solid wire rail 125 is used in conjunction with the flexible coil spring 121. As illustrated in FIG. 17, the solid rail 125 runs down the-lumen 124 of the coil spring 121 providing structural support to the coil spring 121. Multiple rails 125 can also be positioned within the lumen 124 of a coil spring 121. In still another embodiment, the rail 120 is a flexible or substantially rigid elongated rod.

The struts 115 of stent 100 can have substantially any radial thickness that provides them with the needed strength to support a blood vessel while still achieving a low profile that will not damage the vessel as it is deployed. In one example, the struts 115 can have a radial thickness of between about 0.002 inch and about 0.008 inch. In another preferred embodiment, the struts 115 have a radial thickness of between about 0.004 inch and about 0.005 inch. These thicknesses provide the stent 100 with the needed structural and expansion properties to support a vessel and conform to its shape. Additionally, the areas of the curved members 116 must be formed with a greater radial thickness than the struts 115 in order to accommodate the apertures 117. For example, the radial thickness of the curved members 116 can be between about 0.001 inch and about 0.006 inch greater than that of the struts 115. The apertures 117 can have a diameter of about 0.005 inch for receiving the rails 120. Between the rails 120 where expansion occurs, the thickness could be about 0.004 inch. A stent 100 having 0.002 inch thick strut 115 walls could have a curved member 116 with a radial thickness of about 0.009 inch where the rails 120 are woven.

In one embodiment, the process for manufacturing the stent 100 includes the step of providing a hypotube that has a number of small lumens through its wall that form apertures 117. These lumens and the form of each hoop element 110 are laser cut for speed and accuracy. For example, a laser can cut the stent pattern and align the apertures 117 at the peak 112 of the sine wave. Also, the laser provides the hoop elements 110 with a smooth profile. As is understood in the art, the hoop elements 110 should be void of jagged edges because they will damage the vessel and/or not deploy properly. Other known ways of forming these hoops can also be used with the present invention. For example, the stent 100 could be produced using metal extrusion, hot or cold pulling over a fixture of wires, metal injection molding and welding tube assemblies. The supporting rails 120 maintain a relatively smooth profile that has a consistently low friction characteristic in both directions of motion.

The present invention also includes introducing an agent into a body using one of the above-discussed stents. In a preferred embodiment, the agent(s) is carried by one or more of the rail elements 12, 12′ and 120 and released within the body over a predetermined period of time. For example, these stents can deliver one or more known agents, including therapeutic and pharmaceutical drugs, at a site of contact with a portion of the vasculature system or when released from a carrier as is known. These agents can include any known therapeutic drugs, antiplatelet agents, anticoagulant agents, antimicrobial agents, antimetabolic agents and proteins. These agents can also include any of those disclosed in U.S. Pat. No. 6,153,252 to Hossainy et al. and U.S. Pat. No. 5,833,651 to Donovan et al., both of which are hereby incorporated by reference in their entirety. Local delivery of these agents is advantageous in that their effective local concentration is much higher when delivered by the stent than that normally achieved by systemic administration.

The rail elements 12, 12′ and 120, which are relatively inelastic in their transverse strength properties, may carry one or more of the above-referenced agents for applying to a vessel as the vessel moves into contact with the agent carrying rail element(s) 12, 12′ and 120 after deployment of the stent within the vessel. These agents can be applied using a known method such as dipping, spraying, impregnation or any other technique described in the above-mentioned patents that have been incorporated by reference. Applying the agents to the rail elements 12, 12′ and 120 avoids the mechanical disruption that occurs when coated elastic hoop elements are expanded. In this manner drug coatings applied to the stent rail elements 12, 12′ and 120 may be used with hoop elements formed of materials that are otherwise unsuitable for coating.

The use of agent carrying rail elements 12, 12′ and 120 can reduce the complexity and cost of manufacturing agent carrying stents because the rail elements 12, 12′ and 120 can be fabricated in a bulk process and, for example, ribbon coated with one or more agents, including therapeutic drugs, and spooled. The individual agent carrying rail elements 12, 12′ and 120 can be cut to size from a long ribbon of material and introduced through hoop elements to form a stent according to the present invention. Additionally, multiple rail elements cut from different ribbons and carrying the same or different agents can be used in the same stent. For example, if the stent includes three rails elements, the first rail element can carry one agent, the second rail element can carry a second agent that is different from the first agent and the third rail can carry a third agent. The third agent can be the same as one of the agents carried by the other two rail elements or different from the agents carried by the other two rail elements. As a result, the stents according to the present invention permit customization of the agents delivered to the body by allowing different rail elements carrying the same or different agents to be introduced through the hoop elements along the length of a single stent. Additional customizing of a stent can be achieved using rail elements that include different longitudinal sections carrying different agents.

In an alternative embodiment, both the rail elements and the hoop elements of a single stent carry one or more of the above-discussed agents. The agent(s) carried by the hoops can be the same as, or different from, the agents carried by the rail elements. Additionally, the agent(s) carried by one or more of the rail elements can be carried by some of the hoop elements, while the remaining hoop elements and rail elements can carry the same or different agents.

It is contemplated that the various elements of the present invention can be combined with each other to provide the desired flexibility. For example, hoop designs can be altered and various hoop element designs combined into a single stent with/without anyone of the above-discussed rails. Similarly, the number, shape, composition and spacing of the rail elements can be altered to provide the stent with different properties. Additionally, the device can have varying numbers and placement of the bridge elements. The properties of any individual stent would be a function of the design, composition and spacing of the hoops, rails and bridges.

Thus, while there have been shown and described and pointed out fundamental novel features of the present invention as applied to preferred embodiments thereof, it will be understood that various omissions and substitutions and changes in the form and details of the devices illustrated, and in their operation, and in the method illustrated and described, may be made by those skilled in the art without departing from the spirit of the invention as broadly disclosed herein.

We claim:

1. A stent comprising:
a stent element helically wound about an axis; and
at least one rail element extending in parallel with said axis, each said rail element passing alternatingly to the interior and the exterior of said stent element, wherein said stent element is freely movable along and relative to said rail elements.

2. The stent according to claim 1, wherein said at least one rail element includes a plurality of rail elements and at least some of said rail elements include restricting structures at respective ends thereof for restricting movement of said stent element.

3. The stent according to claim 1, wherein said stent element is metallic.

4. The stent according to claim 1, wherein said stent element is made from a resorbable material.

5. The stent according to claim 1, wherein said at least one rail element is metallic.

6. The stent according to claim 1, wherein said at least one rail element is made from one of glass and a polymer.

7. The stent according to claim 1, wherein said stent element is an undulating wire.

8. The stent according to claim 7, wherein said wire has a sawtooth shape.

9. The stent according to claim 7, wherein said wire has a smooth sinusoidal wave shape.

10. The stent according to claim 7, wherein said wire includes diamond shapes distributed along the length thereof.

11. The stent according to claim 9, wherein a peak portion of at least one said sinusoidal wave has a laterally inward narrowed portion to define a restricted passage through which a respective said rail element passes.

12. The stent according to claim 9, wherein a peak portion of at least one said sinusoidal wave has an opening formed therethrough to define a passage through which a respective said rail element passes.

13. The stent according to claim 1 wherein said at least one rail element carries a therapeutic agent and said stent element is free of said agent.

14. The stent according to claim 1 wherein said at least one rail element includes a plurality of rail elements, and at least two of said rail elements carry different therapeutic agents.

15. A stent comprising:
a plurality of generally parallel hoop elements; and
at least one rail element passing alternatingly to the interior and the exterior respective said hoop elements, wherein said hoop elements are freely movable along and relative to said at least one rail element.

16. The stent according to claim 15, wherein said at least one rail element includes a plurality of rail elements and at least some of said rail elements include restricting structures at respective ends thereof for restricting movement of said stent element.

17. The stent according to claim 15, wherein said stent element is metallic.

18. The stent according to claim 15, wherein said stent element is made from a resorbable material.

19. The stent according to claim 15, wherein said at least one rail element is metallic.

20. The stent according to claim 15, wherein said at least one rail element is made from one of glass and a polymer.

21. The stent according to claim 15, wherein said stent element is an undulating wire.

22. The stent according to claim 21, wherein said wire has a sawtooth shape.

23. The stent according to claim 21, wherein said wire has a smooth sinusoidal wave shape.

24. The stent according to claim 21, wherein said wire includes diamond shapes distributed along the length thereof.

25. The stent according to claim 23, wherein a peak portion of at least one said sinusoidal wave has a laterally inward narrowed portion to define a restricted passage through which a respective said rail element passes.

26. The stent according to claim 23, wherein a peak portion of at least one said sinusoidal wave has an opening formed therethrough to define a passage through which a respective said rail element passes.

27. The stent according to claim 15 wherein said at least one rail element carries a therapeutic agent and said hoop elements are free of said agent.

28. The stent according to claim 15 wherein said at least one rail element includes a plurality of rail elements, and at least two of said rail elements carry different therapeutic agents.

29. A stent for deploying within a body, said stent comprising:
first and second terminal ends and a length extending between said terminal ends;
at least one support rail extending between said terminal ends; and
a plurality of circumferentially extending support elements, said support elements each having an aperture that extends along a portion of the length of said stent, said at least one support rail being received within respective apertures so that said support elements are moveable relative to said rail between said terminal ends of said stent.

30. The stent according to claim 29 wherein each said support element includes a first side proximate said first end and a second side proximate said second end, and each opening extends between said first and second sides of a respective one of said support elements.

31. The stent according to claim 29 wherein a first of said support elements is secured to said at least one rail at said first end of said stent; a second of said support elements is secured to said at least one rail at said second end and wherein said remaining support elements are moveable along said at least one rail between said first and second support elements.

32. The stent according to claim 29 wherein said support elements include support hoops.

33. The stent according to claim 32 further comprising a plurality of bridging elements, each said bridging element extending between adjacent support hoops for securing said adjacent support hoops together.

34. The stent according to claim 29 wherein each said support element has an undulating profile including peaks and troughs, and wherein each said aperture is formed in a peak of its respective support element.

35. The stent according to claim 29 wherein said openings extend in a direction substantially parallel to said stent length before deployment of said stent within said body.

36. The stent according to claim 29 wherein said at least one rail is formed of a flexible material.

37. The stent according to claim 29 wherein said at least one rail includes a spring.

38. The stent according to claim 29 wherein said at least one rail is one of a plurality of rails that extend through said support elements, and wherein said rails each comprise a spring.

39. The stent according to claim 38 wherein said rails each comprise a spring.

40. The stent according to claim 39 wherein each said rail extends through an aligned aperture in adjacent support elements.

41. The stent according to claim 29 wherein said at least one support rail carries a therapeutic agent and said support elements are free of said agent.

42. The stent according to claim 29 wherein said at least one rail includes a plurality of rails, and at least two of said rails carry different therapeutic agents.

43. A stent for deploying within a body, said stent comprising:
first and second terminal ends and a length extending between said terminal ends;
at least one support rail extending between said terminal ends; and
a plurality of circumferentially extending support elements, said support elements each having an opening for receiving the at least one support rail so that said support elements are moveable relative to said rail between said terminal ends of said stent and a portion of the stent can longitudinally collapse when positioned within a vessel.

44. The stent according to claim 43 wherein said support rail comprises a spring.

45. The stent according to claim 44 wherein said at least one support rail is one of a plurality of support rails positioned circumferentially around said stent.

46. The stent according to claim 43 wherein said openings extend through said support elements and extend in a direction parallel the length of the stent.

47. The stent according to claim 43 wherein said openings are formed by laterally inward narrowed portions of respective support elements that define restricted passages through which said at least one support rail passes.

48. The stent according to claim 43 wherein a therapeutic agent is carried by said at least one support rail.
